SEQUENCE LISTING (54 sequences)

SEQ ID NO: 1 - DNA, 20 bp, Artificial Sequence
  IL-6 forward primer
agttgccttcttgggactga

SEQ ID NO: 2 - DNA, 20 bp, Artificial Sequence
  IL-6 reverse primer
tccacgatttcccagagaac

SEQ ID NO: 3 - DNA, 20 bp, Artificial Sequence
  IL-1beta forward primer
accatggcacattctgttca

SEQ ID NO: 4 - DNA, 20 bp, Artificial Sequence
  IL-1beta reverse primer
tgcaggctatgaccaattca

SEQ ID NO: 5 - DNA, 20 bp, Artificial Sequence
  TNFalpha forward primer
ctgggacagtgacctggact

SEQ ID NO: 6 - DNA, 20 bp, Artificial Sequence
  TNFalpha reverse primer
gcacctcagggaagagtctg

SEQ ID NO: 7 - DNA, 20 bp, Artificial Sequence
  IGF1 forward primer
aggggaacaggaggaggtaa

SEQ ID NO: 8 - DNA, 20 bp, Artificial Sequence
  IGF1 reverse primer
agtgaggactgccttgcttc

SEQ ID NO: 9 - DNA, 20 bp, Artificial Sequence
  IGF2 forward primer
gccctcctggagacatactg

SEQ ID NO: 10 - DNA, 20 bp, Artificial Sequence
  IGF2 reverse primer
cgtttggcctctctgaactc

SEQ ID NO: 11 - DNA, 21 bp, Artificial Sequence
  TLR2 forward primer
tctgggcagtcttgaacattt

SEQ ID NO: 12 - DNA, 21 bp, Artificial Sequence
  TLR2 reverse primer
agagtcaggtgatggatgtcg

SEQ ID NO: 13 - DNA, 21 bp, Artificial Sequence
  TLR4 forward primer
gcaatgtctctggcaggtgta

SEQ ID NO: 14 - DNA, 21 bp, Artificial Sequence
  TLR4 reverse primer
caagggataagaacgctgaga

SEQ ID NO: 15 - DNA, 20 bp, Artificial Sequence
  OC forward primer
gcagcttggtgcacacctag

SEQ ID NO: 16 - DNA, 20 bp, Artificial Sequence
  OC reverse primer
ggagctgctgtgacatccat

SEQ ID NO: 17 - DNA, 22 bp, Artificial Sequence
  OPG forward primer
gtggtgcaagctggaaccccag

SEQ ID NO: 18 - DNA, 23 bp, Artificial Sequence
  OPG reverse primer
aggcccttcaaggtgtcttggtc

SEQ ID NO: 19 - DNA, 17 bp, Artificial Sequence
  MMP-9 forward primer
ccatgagtccctggcag

SEQ ID NO: 20 - DNA, 19 bp, Artificial Sequence
  MMP-9 reverse primer
agtatgtgatgttatgatg

SEQ ID NO: 21 - DNA, 25 bp, Artificial Sequence
  RANKL forward primer
cgctctgttcctgtactttcgagcg

SEQ ID NO: 22 - DNA, 25 bp, Artificial Sequence
  RANKL reverse primer
tcgtgctccctcctttcatcaggtt

SEQ ID NO: 23 - DNA, 21 bp, Artificial Sequence
  RANK forward primer
cacagacaaatgcaaaccttg

SEQ ID NO: 24 - DNA, 24 bp, Artificial Sequence
  RANK reverse primer
gtgttctggaacctatcttcctcc

SEQ ID NO: 25 - DNA, 20 bp, Artificial Sequence
  NPY forward primer
tgtttgggcattctggctga

SEQ ID NO: 26 - DNA, 20 bp, Artificial Sequence
  NYY reverse primer
ttctgggggcgttttctgtg

SEQ ID NO: 27 - DNA, 28 bp, Artificial Sequence
  NPY1receptor forward primer
ctcgctggttctcatcgctgtggaacgg

SEQ ID NO: 28 - DNA, 22 bp, Artificial Sequence
  NPY1receptor reverse primer
gcgaatgtatatcttgaagtag

SEQ ID NO: 29 - DNA, 20 bp, Artificial Sequence
  NPY2receptor forward primer
tcctggattcctcatctgag

SEQ ID NO: 30 - DNA, 20 bp, Artificial Sequence
  NPY2receptor reverse primer
ggtccagagcaatgactgtc

SEQ ID NO: 31 - DNA, 20 bp, Artificial Sequence
  Leptin forward primer
ttcacacacgcagtcggtat

SEQ ID NO: 32 - DNA, 20 bp, Artificial Sequence
  Leptin reverse primer
ctcaaagccaccacctctgt

SEQ ID NO: 33 - DNA, 21 bp, Artificial Sequence
  GAPDH forward primer
gtcagcaatgcatcctgcacc

SEQ ID NO: 34 - DNA, 21 bp, Artificial Sequence
  GAPDH reverse primer
tcattgagagcaatgccagcc

SEQ ID NO: 35 - DNA, 20 bp, Artificial Sequence
  c-Jun forward primer
tcccctatcgacatggagtc

SEQ ID NO: 36 - DNA, 20 bp, Artificial Sequence
  c-Jun reverse primer
tgagttggcacccactgtta

SEQ ID NO: 37 - DNA, 20 bp, Artificial Sequence
  Jun D forward primer
cgaccagtacgcagttcctc

SEQ ID NO: 38 - DNA, 20 bp, Artificial Sequence
  JunD reverse primer
aactgctcaggttggcgtag

SEQ ID NO: 39 - DNA, 20 bp, Artificial Sequence
  c-Fos forward primer
ccagtcaagagcatcagcaa

SEQ ID NO: 40 - DNA, 20 bp, Artificial Sequence
  c-Fos reverse primer
aagtagtgcagcccggagta

SEQ ID NO: 41 - DNA, 20 bp, Artificial Sequence
  Fra-1 forward primer
agagctgcagaagcagaagg

SEQ ID NO: 42 - DNA, 20 bp, Artificial Sequence
  Fra-1 reverse primer
caagtacgggtcctggagaa

SEQ ID NO: 43 - DNA, 20 bp, Artificial Sequence
  Fra-2 forward primer
atccacgctcacatccctac

SEQ ID NO: 44 - DNA, 20 bp, Artificial Sequence
  Fra-2 reverse primer
gtttctctccctccggattc

SEQ ID NO: 45 - DNA, 20 bp, Artificial Sequence
  NFATc1 forward primer
gggtcagtgtgaccgaagat

SEQ ID NO: 46 - DNA, 20 bp, Artificial Sequence
  NFATc1 reverse primer
ggaagtcagaagtgggtgga

SEQ ID NO: 47 - DNA, 20 bp, Artificial Sequence
  ALP forward primer
gctgatcattcccacgtttt

SEQ ID NO: 48 - DNA, 20 bp, Artificial Sequence
  ALP reverse primer
ctgggcctggtagttgttgt

SEQ ID NO: 49 - DNA, 20 bp, Artificial Sequence
  OPN forward primer
cgatgatgatgacgatggag

SEQ ID NO: 50 - DNA, 22 bp, Artificial Sequence
  OPN reverse primer
tggcatcaggatactgttcatc

SEQ ID NO: 51 - DNA, 20 bp, Artificial Sequence
  COLLI forward primer
acgtcctggtgaagttggtc

SEQ ID NO: 52 - DNA, 20 bp, Artificial Sequence
  COLLI reverse primer
cagggaagcctctttctcct

SEQ ID NO: 53 - DNA, 20 bp, Artificial Sequence
  GAPDH forward primer
aactttggcattgtggaagg

SEQ ID NO: 54 - DNA, 20 bp, Artificial Sequence
  GAPDH reverse primer
acacattgggggtaggaaca

(19) United States
(12) Patent Application Publication
(10) Pub. No.: US 2010/0333217 A1
(43) Pub. Date: Dec. 30, 2010

(54) SCREENING METHOD FOR THE COMPOSITION FOR PREVENTION OR TREATMENT OF OSTEOPOROSIS AND METABOLIC BONE DISEASE USING TALLYHO/JNGJ MOUSE

(75) Inventors: Myung Ae Bae, Taejeon-si (KR); Hyae Gyeong Cheon, Taejeon-si (KR); Sung Eun Yoo, Chungcheongnam-do (KR); Hee Youn Kim, Taejeon-si (KR); Won Hoon Jung, Taejeon-si (KR); Sang Dal Rhee, Taejeon-si (KR); Doh Yeon Ahn, Taejeon-si (KR)

Correspondence Address: KLARQUIST SPARKMAN, LLP, 121 SW SALMON STREET, SUITE 1600, PORTLAND, OR, 97204, US

(73) Assignee: KOREA RESERACH INSTITUTE OF CHEMICAL TECHNOLOGY, Taejeon-si (KR)

(21) Appl. No.: 12/680,477

(22) PCT Filed: Apr. 23, 2008
(86) PCT No.: PCT/KR2008/002286

§ 371 Date: Mar. 26, 2010

(30) Foreign Application Priority Data
Sep. 28, 2007 (KR) 10-2007-0098323

(51) Int. Cl. G01N 33/00; G01N 33/50; C12Q 1/68

(52) U.S. Cl. 800/3; 436/86; 435/6

(57) ABSTRACT

Provided is a method for screening a composition for preventing or treating an osteoporosis and a metabolic bone disease using a TALLYHO/JngJ mouse, and more particularly, to a method for screening a compound effective in preventing and treating a disease caused by abnormalities of a bone metabolism including an osteoporosis, through assessment of a bone regeneration activity, such as acceleration of osteoblast differentiation, inhibition of osteoclast differentiation, variation of cytokine in a serum, as well as a bone mineral density and a bone mineral content in a femur, using a TALLYHO/JngJ mouse instead of a ovariectomized animal generally used in estimating the efficacy of a new osteoporosis medicine in an animal. The screening method has a simple and stable experiment procedure, and can be used in developing an agent for treating an osteoporosis of men and improving the bone mineral content. Therefore, the method can be used in estimating an indirect efficacy on the metabolic bone disease and the osteoporosis.

### TECHNICAL FIELD

The present disclosure relates to a method for screening a composition for preventing or treating an osteoporosis and a metabolic bone disease using a TALLYHO/JngJ (hereinafter referred to as TH) mouse, and more particularly, to a method for screening a compound effective in preventing and treating a disease caused by abnormalities of a bone metabolism including an osteoporosis, through assessment of a bone regeneration activity, such as acceleration of osteoblast differentiation, inhibition of osteoclast differentiation, variation of cytokine in a serum, as well as a bone mineral density and a bone mineral content in a femur, using a TH mouse instead of a generally used ovariectomized animal.

### BACKGROUND ART

With the development in medical science and genetic engineering, it is estimated that the elderly people over the age of 60 will comprise 25 percent of the population in the 21st century. Accordingly, research and development on senile diseases are growing in social and pharmaceutical industrial importance. Osteoporosis is a representative senile disease, and the number of patients suffering from the osteoporosis significantly increases particularly in industrial nations with a large number of elderly people.

A bone tissue in a human body is a dynamic organ formed by bone remodeling such as bone formation of osteoblast and bone resorption of osteoclast, which is unceasingly repeated throughout life. Balance between the bone formation and the bone resorption allows the bone mineral content in the bone tissue and the function of the bone tissue to keep their normal state. The bone tissue is necessary to maintain a life because it serves as a supporting tissue for supporting human body and serves to preserve important organs and hematopoietic hepatocytes so as to provide blood cells. The osteoporosis is quickly developed by a fracture due to bone loss which is caused by removal of calcium from the bone which is occurred by the balance between the bone resorption and the bone formation is broken to accelerate the bone resorption. According to a previous research report, recently, even young persons are attacked with the osteoporosis due to lack of calcium intake caused by an unbalanced diet and the like, as well as elder persons over the age of 65 susceptible to a senile osteoporosis and middle-aged women susceptible to a postmenopausal osteoporosis due to lack of sex hormone. The dangers of the osteoporosis are increased in persons suffering from hypertension, hyperlipidemia, diabetes, liver disease, renal failure, thyroid disease, cancer, or sexual dysfunction, and persons taking steroids or stomach and bowel medicines for a long time, persons taking much alcohols, tobaccos or coffees, persons taking much meats, persons taking little exercises, skinny persons, sedentary workers, persons had an operation on the stomach, persons suffering from lumbago, arthritis or myalgia, long supine persons, persons susceptible to fatigue, and the like. However, the mechanism thereof is not well known yet.

A typical experimental method for developing a therapeutic agent for the osteoporosis is as follows. First, a material for inhibiting activation of the osteoclast or promoting activation of the osteoblast is selected. Then, the efficacy of the material is assessed by measuring the amount of recovered bone mineral content and the amount of recovered bone strength, using an animal developing symptoms of the osteoporosis similar to human beings. A general animal model currently used for the osteoporosis utilizes animals where bone loss was induced through an artificial ovariectomy or a naturally aged SAMP-6 mouse to administer the developed agents thereto and then determine the efficacy of the medicine.

However, current ovariectomized osteoporosis animals require ovariectomy operations for every experiment, thereby increasing experiment time and cost and increasing experimental error due to the failure of the operation. Furthermore, aged models such as SAMP-6 need to be taken care of for a long time in the laboratory and may cause the experimental error. Meanwhile, recently, advanced pharmaceutical companies decided that it is necessary to resolve the problems, such as safety, absortionability and price, of the existing osteoporosis medicines to develop a new competitive osteoporosis medicine. Accordingly, they are making huge investments in researches for finding a new initial target point and in developments of a new medicine using the same, and they are taking interest in the combination therapeutic strategies (combination of an anti-resorptive agent and an anabolic agent). That is, they determined that it is the time to establish the research strategy for developing a compound for inhibiting the activation of the osteoclast and promoting the activation of the osteoblast at the same time, which is safe and has excellent absorptionability, by utilizing new initial target points and a variety of new approaches.

The TH mouse was recently established by Jackson Laboratory, USA, through selective breeding of transformed animals developing diabetes by type 2 diabetes model due to a new Darwin gene. The TH mouse was reported in 2001 that only male mice develop diabetes between 10 week ages and 14 week ages, and they show abdominal obesity although it is not serious. However, there has been no report with respect to the osteoporosis.

### DISCLOSURE OF THE INVENTION

### Technical Problem

An aspect of the present invention provides an effective screening method using a TH mouse as a new disease animal model for developing a therapeutic medicine for osteoporosis caused by a variety of factors such as sex hormone, aging, inflammation, diabetes and the like.

### Technical Solution

The inventors found that a TH mouse develops osteoporosis, and thus found that it is possible to screen an ideal osteoporosis medicine, i.e., a therapeutic agent for improving a bone mineral density or a composition for preventing or treating a metabolic bone disease using a mechanism for causing the osteoporosis and the TH mouse.

Exemplary embodiments of the present invention provide a method for screening an agent for treating or preventing an osteoporosis and a metabolic bone disease, including:

1) a step of administering a candidate material for an agent for treating or preventing the osteoporosis and the metabolic bone disease to a male TH mouse;

2) a step of measuring an index related to the osteoporosis and the metabolic bone disease from the male TH mouse administered with the candidate material of the step 1); and

3) a step of comparing the index of the male TH mouse administered with the candidate material and that of a control group which is not administered with the candidate material to determine whether the candidate material varies the index with significance.

Hereinafter, the present invention will be described in detail.

An exemplary embodiment provides a method for screening an agent for treating or preventing an osteoporosis and a metabolic bone disease using a TH mouse.

The weights and the feed intakes of the TH mouse and the control mouse were measured, and the result told us that the feed intake of the TH mouse was greater than that of the control mouse, and the weight of the TH mouse was increased with significance (see FIG. 1).

The femurs and the skulls of the TH mouse and the control mouse were analyzed, and the result told us that the femur of the male TH mouse was reduced with significance in a bone mineral density and a bone mineral content compared with the control mouse, and the skull of the 8-week-old male TH mouse was reduced in the bone mineral density and the bone thickness compared with the control mouse.

The bone marrow was separated from the femur of the TH mouse and then the differentiations of the osteoblast and the osteoclast were induced. As a result, giant multinucleate cells were observed in the osteoblast culture group, and they were identified as osteoclasts (see FIG. 4).

The expression level of the genes related to the differentiation of the osteoblast and the osteoclast was examined. An osteoprotegerin (OPG) playing an important role in the bone formation was reduced in the TH mouse. However, a receptor activator of NF-κ B ligand (RANKL) causing the bone resorption was significantly increased in the TH mouse. In addition, IL-6, which is one of cytokines playing an important role in the bone loss was increased in the TH mouse (see FIG. 5).

Further, in order to determine whether the lack of the bone differentiation in the TH mouse is a posteriori or a priori, the shape of the osteoblast separated from a skull of a 1-day-old mouse was examined, and the variation of the expression of the genes related to the bone differentiation was also examined. The examination on the expression of the genes related to the differentiation of the osteoblast and the differentiation transcription genes in the cell, which is cultured for 8 days, showed a significant decrease in the gene expression of the transcription factors, such as Fra2, NF-AT1, JunD, and Fos, which control the differentiation of the osteoblast (see FIGS. 6 to 14).

From the above mentioned results, it can be seen that the male TH mouse of an age of 4 weeks shows features similar to the ovariectomized mouse model.

Hence, the inventors further investigated the effects of alendronate and parathyroid hormone (PTH), which showed the effect of bone formation in the ovariectomized mouse model, in the male TH mouse. The result was similar to the ovariectomized osteoporosis model mouse (see FIGS. 15 to 24).

As a result, it can be concluded that the male TH mouse can be used usefully as a new animal model in the development of the osteoporosis medicine.

When using the TH mouse, the artificial ovariectomy operation is not required because of the bone loss induced by the excessive amount of IL-6 in the blood serum and the problem of the complex bone-related gene expression. In addition, the ratio of OPG to RANKL, which is a representative feature of the osteoporosis, is significantly small. Therefore, the male TH mouse can be used as a useful natural disease model in the development of the new medicine for treating and preventing diseases such as a pre- or post-menopausal osteoporosis of women, a senile osteoporosis, a osteoporosis of men, an osteoporosis after a variety of implantations, an osteoporosis after a variety of surgeries including a heart valve surgery and a gastrectomy, a secondary osteoporosis caused by an osteomalacia and a steroid, and an inflammation including an osteoarthritis.

Another exemplary embodiment provides a screening method for an agent for treating or preventing an osteoporosis and a metabolic bone disease, including:

1) a step of administering a candidate material for an agent for treating or preventing the osteoporosis and the metabolic bone disease to a male TH mouse;

2) a step of measuring an index related to the osteoporosis and the metabolic bone disease from the male TH mouse administered with the candidate material of the step 1); and

3) a step of comparing the index of the male TH mouse administered with the candidate material and that of a control group which is not administered with the candidate material to determine whether the candidate material varies the index with significance.

The candidate material includes, for example, a peptide, a protein, a nonpeptide compound, a synthetic compound, a fermentation product, a cell extract, a plant extract, an animal texture extract, or a blood plasma. Such a compound may be a new compound or a well-known compound. The candidate material may form a salt thereof. The salt of the candidate material includes an acid (e.g., an inorganic acid) or a base (e.g., an organic acid, etc) which is physiologically acceptable, and is preferably a physiologically acceptable acid-added salt. For example, a salt of the inorganic acid (e.g., hydrochloric acid, phosphoric acid, hydrobromic acid, sulfuric acid, etc), or a salt of the organic acid (e.g., acetic acid, formic acid, propionic acid, fumaric acid, maleic acid, succinic acid, tartaric acid, citric acid, malic acid, oxalic acid, benzoic acid, methanesulfonic acid, bezenesulfonic acid, etc) may be used as the salt.

The administration method for the candidate material may be appropriately selected according to the symptoms of the experiment animal, the characteristics of the candidate material, and the like. The administration method includes, for example, oral administration, intravenous injection, swabbing, subcutaneous administration, intracutaneous administration, intraperitoneal administration or the like. In addition, the dose of the candidate material may also be appropriately selected according to the administration method, the characteristics of the candidate material, and the like.

The index related to the osteoporosis of the step 2) includes, for example, increase of the bone mineral density and the bone mineral content of the femur, increase of the thickness and the bone mineral density of the skull, increase of OPG or decrease of IL-6 in the blood serum, decrease of RANKL, increase of the expressions of Fra2, NF-AT1, JunD and Fos genes which are transcription factors for controlling the differentiation of the osteoblast, and increase of expressions of alkaline phosphatase and COLL I genes which are differentiation factors of the osteoblast.

By comparing the above mentioned indexes of the male TH mouse administered with the candidate material and the control mouse administered with no candidate material, it can be determined whether the candidate material affects the index or not. As such, it is possible to screen the agent for treating or preventing the osteoporosis and the metabolic bone disease.

### Advantageous Effects

The screening method using the TH mouse according to the present invention has the following effects in comparison to the screening method using the typical ovariectomized osteoporosis animal. The experiment procedure is simple and stable. The screening method using the TH mouse may also be used usefully in predicting indirect efficacy on the metabolic bone disease as well as the osteoporosis. The male TH mouse according to the present invention can be used as a new animal model in the development of the medicine for osteoporosis symptom caused by a variety of causes such as a senile osteoporosis, an osteoporosis due to an inflammation, an osteoporosis due to inherited components, as well as osteoporosis symptoms of menopausal women.

### BRIEF DESCRIPTION OF THE DRAWINGS

FIG. 1 illustrates graphs of weights versus week ages of control mice and TH mice according to an embodiment of the present invention.

FIG. 2(a) illustrates graphs of bone mineral densities (BMD) versus week ages of control mice (C57BL/6) and TH mice according to an embodiment of the present invention; and FIG. 2(b) illustrates graphs of bone mineral contents (BMC) versus week ages of control mice (C57BL/6) and TH mice according to an embodiment of the present invention.

FIG. 3 illustrates graphs showing thicknesses of skulls of 8-week-old TH mice according to an embodiment of the present invention, the thicknesses each being measured by a micro CT after separating the skull.

FIGS. 4A and 4B illustrate photographs of cell groups and expression of genes, which are obtained after separating osteoblasts from 1-day-old C57BL/6 mice and TH mice according to an embodiment of the present invention and then culturing them for 8 days.

FIG. 5 illustrates photographs showing expression levels of cytokines and genes related to differentiations of an osteoblast and an osteoclast which are cultured after separating bone marrows from femurs of respective sexes and week ages of TH mice according to an embodiment of the present invention.

FIGS. 6 to 14 illustrate expressions of ALP, OP, Coll, c-Jun, c-Fos, jun-D, fra-1, fra-2 and NFATc1, respectively, which are genes related to differentiation of an osteoblast and an osteoclast, from cells generated after culturing bone marrows separated from femurs of 8-week-old TH mice according to an embodiment of the present invention.

FIGS. 15 to 19 illustrate results of inhibiting bone loss and promoting bone formation after treating 4-week-old TH mice according to an embodiment of the present invention with alendronate, which is widely administered clinically, for 4 weeks, with graphs of bone mineral densities of femurs (FIG. 15), bone mineral contents of femurs (FIG. 16), thicknesses of skulls (FIG. 17), and bone mineral densities of skulls (FIG. 18), and histomorphological scan images (FIG. 19).

FIGS. 20 and 21 illustrate results of inhibiting bone loss and promoting bone formation after treating 4-week-old TH mice according to an embodiment of the present invention with alendronate, which is widely administered clinically, for 4 weeks, with graphs showing IL-6 level of blood serums (FIG. 20), and photographs showing the effect thereof on expression of genes related to differentiations of an osteoblast and an osteoclast from cells generated after culturing bone marrows separated from femurs of mice (FIG. 21).

FIGS. 22 to 24 illustrate results of inhibiting bone loss and promoting bone formation after subcutaneously administering a parathyroid hormone, which is recently permitted for clinical use, to 8-week-old TH mice according to an embodiment of the present invention for 4 weeks, with graphs of bone mineral densities of skulls (FIG. 22) and bone mineral contents (FIG. 23), and histomorphological scan images (FIG. 24).

### BEST MODE FOR CARRYING OUT THE INVENTION

Hereinafter, exemplary embodiments will be described in detail with reference to the accompanying drawings.

However, it should be understood that the description of the exemplary embodiments is merely illustrative and that it should not be taken in a limiting sense.

### Example 1

### Weight Variation and Feed Intake According to Week Age of TH Mouse

Control mice (C57BL/6) and TH mice (Jackson Laboratory, bar harbor, USA) were freely fed, and their weights were measured every week from 4 week age to 20 week age after delectation.

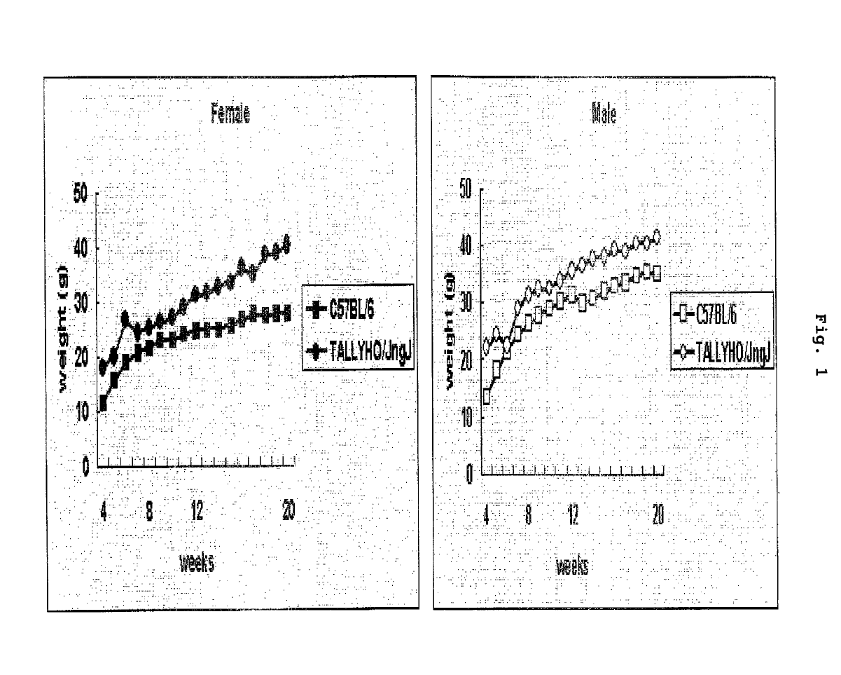

As shown in FIG. 1, the TH mice were increased in weight with significance compared with the control mice. The feed intake was slightly greater in the TH mice than in the control mice.

### Example 2

### Bone Mineral Density and Bone Mineral Content in Femur of TH Mouse According to Week Age and Sex Thereof

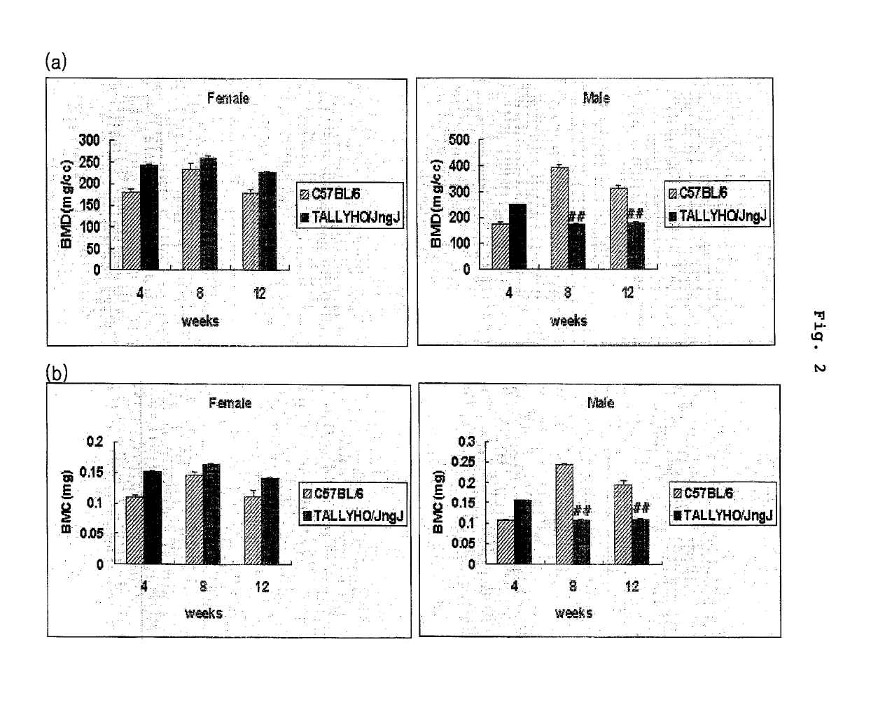

Control mice (C57BL/6) and TH mice were freely fed, and their bone mineral densities were measured every week from 4 week age to 20 week age after delectation. The mice were anesthetized with Avertin (2,2,2-tribromoethanol) to keep them alive while measuring their bone mineral densities. Then, scanning was performed 400 times at a thickness of 45 using eXplore Locus micro-CT (GE Healthcare, USA) which is devised as a tomographic apparatus for small animals. Thereafter, the resulting images were reformed using Microview (GE Healthcare, USA) to obtain the final bone mineral density (BMD) and the final bone mineral content (BMC) (see FIG. 2).

As shown in FIG. 2, interestingly, in comparison to the control mice, BMD (FIG. 2(a)) and BMC (FIG. 2(b)) of the male TH mice were decreased with significance continuously after 4 week age, while those of the female TH mice were decreased after 20 week age.

This is probably because of the natural specific bone loss of the male TH mice. The TH mice showed the same symptoms as the ovariectomized mice without the artificial ovariectomy. Therefore, this result shows the possibility of the male TH mice as a new osteoporosis animal model.

### Example 3

### Variation of Bone Mineral Density and Bone Thickness in Skull of TH Mouse

Control mice (C57BL/6) and TH mice were fed freely. 8-week-old mice which showed bone loss based on male TH mice were anesthetized and then sacrificed to extract their skulls. Thereafter, the bone thicknesses and the bone mineral densities of the extracted skulls were measured as described in Example 2, using a tomographic apparatus (micro-CT) for animals.

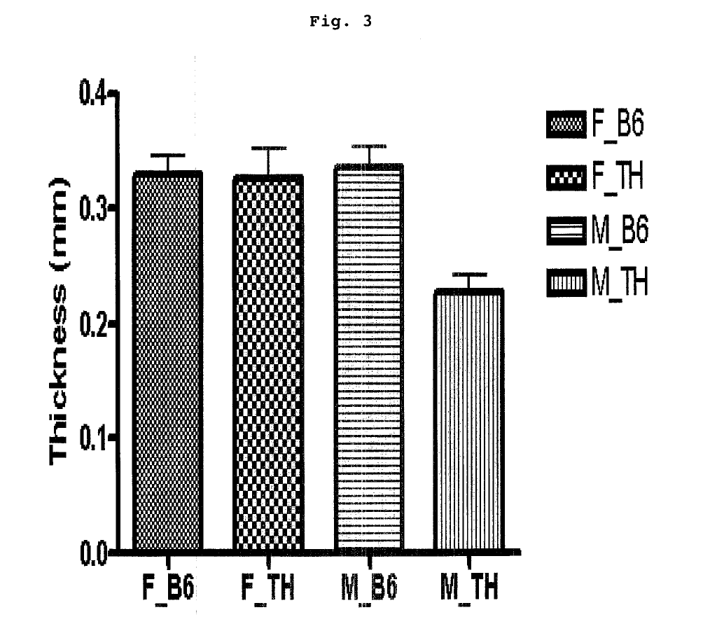

As shown in FIG. 3, the bone mineral densities and the bone thicknesses in the skulls of the male TH mice were decreased compared with the control mice, which was similar to the bone losses of their femurs. This is probably because of the natural specific bone loss of the male TH mice. This result shows the possibility of a new index for an osteoporosis animal model according to the experiment procedure newly proposed by the present invention.

### Example 4

### Induction of Differentiation of Osteoblast and Osteoclast of TH Mouse Cultured After Separation of Bone Marrow from Femur of TH Mouse

This example was performed to examine the possibility of TH mice as an osteoporosis animal model. Bone marrows were separated from control mice (C57BL/6) and TH mice, and then cells were cultured for 1 day. After removing floating cells, the cells adsorbed to an incubator were differentiated to osteoblasts for 8 days while replacing an alpha-MEM medium added with ascorbic acid and beta-glycerolphosphate which are osteoblast differentiation accelerators, and 10% bovine serum, with a new alpha-MEM medium every three days.

Using the plates cultured for 8 days, an alkaline phosphatase (ALP) staining and a tartrate-resistant acid phosphatase (TRAP) staining were performed to measure the differentiation degree of the osteoblast and the osteoclast, respectively. The ALP staining was performed as follows. The medium in the culture plate was removed and washing was performed twice with 1×PBS. The cells were fixed with 2% paraformaldehyde (Sigma, USA) at an ambient temperature for 10 minutes. The fixed cells were added with diazonium salt solution mixed with equivalent amounts of sodium nitrate solution (Sigma, USA) and FRV-alkaline phosphatase solution (Sigma, USA) and reacted at an ambient temperature for 20 minutes to examine the activity of the stained cells. The TRAP staining was performed as follows. Washing was performed twice with 1×PBS. 4% formalin solution (Sigma, USA) was added to fix the cells at an ambient temperature for 10 to 15 minutes. The TRAP staining solution (50 mM acetate buffer, 30 mM sodiotartrate, 0.1 mg/ml naphthol, 0.1% triton X-100, 0.03% fast red violet, Sigma, USA) was added to react with the cells at 37° C. in a darkness for 30 minutes to 1 hour. The activity was measured by counting only giant multinucleate cells merged with three or more nuclei to assess the differentiation amount of the osteoclast.

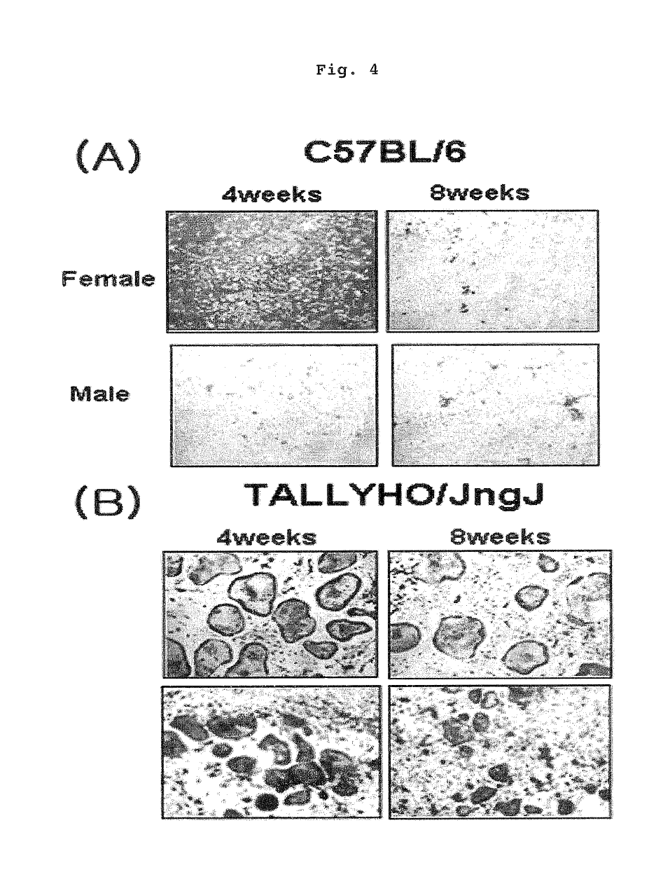

Consequently, as shown in FIG. 4(A), contrary to the osteoblast of B6 group, the osteoblast separated from the TH mouse was slightly stained. Meanwhile, as shown in FIG. 4(B), in an experiment group for inducing differentiation of osteoblast, a giant multinucleate cell was interestingly observed in the osteoblast culture group separated from the TH mice.

Considering that one of the major factors in the osteoporosis is the osteoclast activation faster than the osteoblast activation, it can be seen from the above mentioned result that the bone loss occurred in the TH mice.

### Example 5

### Variation of Genes in Osteoblast and Osteoclast Separated from Bone Marrow of 8-Week-Old Mice

After separating mRNA from the cell of FIG. 4, the expression levels of the genes related to the differentiation of osteoblast and osteoclast and cytokines were analyzed. The total RNA was obtained from the Extraction Kit (Intron, Seongnam, Korea) according to the manufacturer's protocol. The separated RNA was weighed by measuring the absorbance. The reverse transcription PCR (RT-PCR) was performed using primers listed in Table 1 under the following conditions to examine the expression level of each of the genes.

TABLE 1

| Target gene | Forward (5′-3′) | Reverse (5′-3′) |
|---|---|---|
| IL-6 | AGTTGCCTTCTTGGGACTGA | TCCACGATTTCCCAGAGAAC |
| IL-1beta | ACCATGGCACATTCTGTTCA | TGCAGGCTATGACCAATTCA |
| TNFalpha | CTGGGACAGTGACCTGGACT | GCACCTCAGGGAAGAGTCTG |
| IGF1 | AGGGGAACAGGAGGAGGTAA | AGTGAGGACTGCCTTGCTTC |
| IGF2 | GCCCTCCTGGAGACATACTG | CGTTTGGCCTCTCTGAACTC |
| TLR2 | TCTGGGCAGTCTTGAACATTT | AGAGTCAGGTGATGGATGTCG |
| TLR4 | GCAATGTCTCTGGCAGGTGTA | CAAGGGATAAGAACGCTGAGA |
| OC | GCAGCTTGGTGCACACCTAG | GGAGCTGCTGTGACATCCAT |
| OPG | GTGGTGCAAGCTGGAACCCCAG | AGGCCCTTCAAGGTGTCTTGGTC |
| MMP-9 | CCATGAGTCCCTGGCAG | AGTATGTGATGTTATGATG |
| RANKL | CGCTCTGTTCCTGTACTTTCGA | TCGTGCTCCCTCCTTTCATCAGGT |
| | GCG | T |
| RANK | CACAGACAAATGCAAACCTTG | GTGTTCTGGAACCTATCTTCCTCC |
| NPY | TGTTTGGGCATTCTGGCTGA | TTCTGGGGGCGTTTTCTGTG |
| NPY1receptor | CTCGCTGGTTCTCATCGCTGTG | GCGAATGTATATCTTGAAGTAG |
| | GAACGG | |
| NPY2receptor | TCCTGGATTCCTCATCTGAG | GGTCCAGAGCAATGACTGTC |
| Leptin | TTCACACACGCAGTCGGTAT | CTCAAAGCCACCACCTCTGT |
| GAPDH | GTCAGCAATGCATCCTGCACC | TCATTGAGAGCAATGCCAGCC |

Reverse Transcription System (Promega, USA) was added with total RNA 1 μg, 10 mM dNTP 2 μl, 100 pmole Oligo dT 1 μl, 10×buffer 2 μl, 25 mM MgCl₂ 4 μl, RNase inhibitor 0.2 μl, AMV RTase 0.2 μl, and DEPC-treated water, and cultured at 42° C. for 60 minutes and at 72° C. for 10 minutes to prepare cDNA. Then, the reverse transcripted cDNA sample was amplified using primer pairs specific to respective target genes. The PCR was repeated 25-30 times after denaturation at 95° C. for 5 minutes. In specific, the PCR was performed under the condition of denaturation at 95° C. for 30 seconds, primer annealing at 55° C. (NPY1receptor), 56° C. (IL-6, IL-1beta, TNFalpha, TLR2, TLR4, OC, MMP-9, GAPDH), 57° C. (OPG), 58° C. (RANK), 60° C. (IGF1, IGF2, RANKL, NPY, NPY2receptor) and 62° C. (Leptin) for 30 seconds, and extension reaction at 72° C. for 30 seconds. The PCR product was electrophoresed in an 1.5% agarose gel and stained with GelRed. The result was photographed with GelDoc (BioRad, USA).

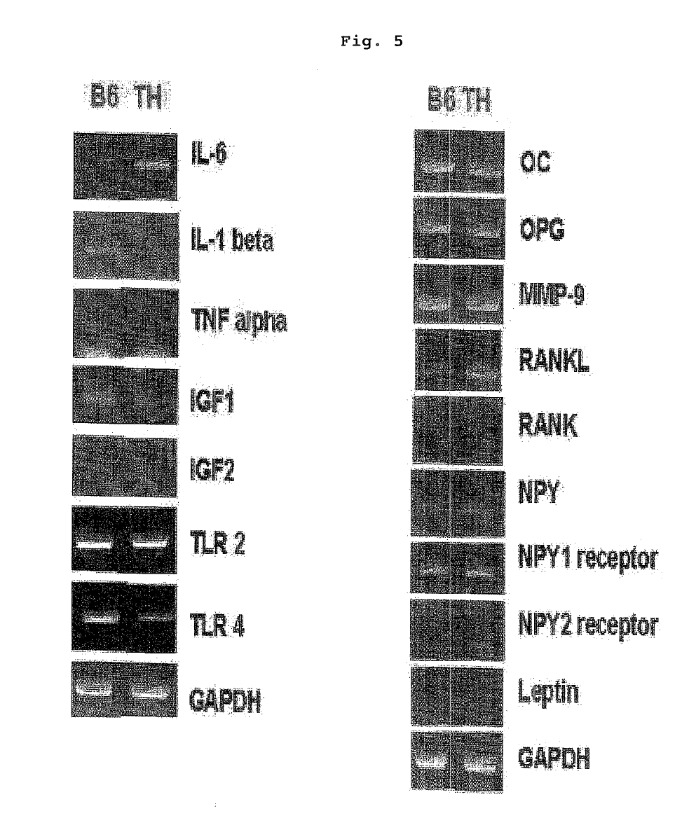

Resultantly, as shown in FIG. 5, it was observed that, in the TH mouse, the OPG playing an important role in the bone formation was decreased, however, the RANKL causing the bone resorption was significantly increased. In addition, it was also observed that the IL-6, which is one of cytokines playing an important role in the bone loss, was increased in the TH mouse.

### Example 6

### Variation of Genes in Osteoblasts Separated from Skulls of 1-Day-Old Mice

In order to determine whether the lack of the bone differentiation in the TH mouse is a posteriori or a priori, skulls were separated from 1-day-old mice, and cultured for 8 days in alpha-MEM media added with betaglycerophosphate and ascorbic acid which are osteoblast differentiation induction factors. Using real time reverse transcription PCR (Real time PCR, Rotor-Gene 300 real-time DNA detection system; Corbett Research, Sydney, Australia), variations of the genes related to the bone differentiation were examined. Total RNA was separated and cDNA was synthesized as described in Example 5. To investigate the expression of each of the genes, 20 pmol of each primer listed in the Table 2 was mixed with cDNA and SYBR Green Master Kit (Stratagene, USA), and PCR was performed 40 times after the dinaturation at 94° C. for 10 minutes under the condition of denaturation at 94° C. for 40 seconds, primer annealing at 60° C. for 40 seconds, and extension reaction at 72° C. for 1 minute. A standard curve was obtained using glyceraldehyde-3-phosphate (GAPDH), and mRNA was obtained by subtracting Ct (threshold cycle) of each gene from GAPDH among calculated Ct values and calculating delta CT value with a formula, 2.0−(Ct). Finally, the expression levels of the mice were shown relatively based on the male TH mice.

TABLE 2

| Target | | |
|---|---|---|
| gene | Forward (5′-3′) | Reverse (5′-3′) |
| c-Jun | TCCCCTATCGACATGGAGTC | TGAGTTGGCACCCACTGTTA |
| Jun D | CGACCAGTACGCAGTTCCTC | AACTGCTCAGGTTGGCGTAG |
| c-Fos | CCAGTCAAGAGCATCAGCAA | AAGTAGTGCAGCCCGGAGTA |
| Fra-1 | AGAGCTGCAGAAGCAGAAGG | CAAGTACGGGTCCTGGAGAA |
| Fra-2 | ATCCACGCTCACATCCCTAC | GTTTCTCTCCCTCCGGATTC |
| NFATc1 | GGGTCAGTGTGACCGAAGAT | GGAAGTCAGAAGTGGGTGGA |
| ALP | GCTGATCATTCCCACGTTTT | CTGGGCCTGGTAGTTGTTGT |
| OPN | CGATGATGATGACGATGGAG | TGGCATCAGGATACTGTTCATC |
| COLLI | ACGTCCTGGTGAAGTTGGTC | CAGGGAAGCCTCTTTCTCCT |
| GAPDH | AACTTTGGCATTGTGGAAGG | ACACATTGGGGGTAGGAACA |

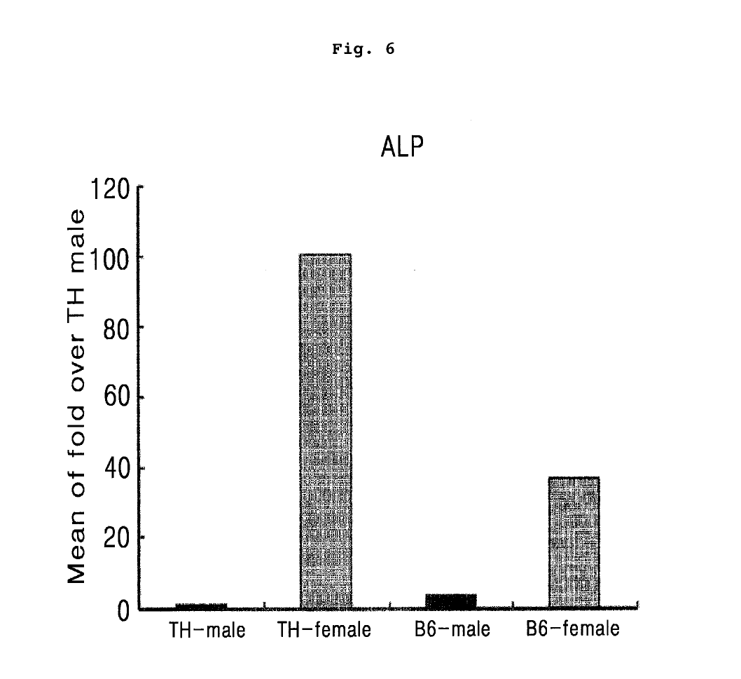

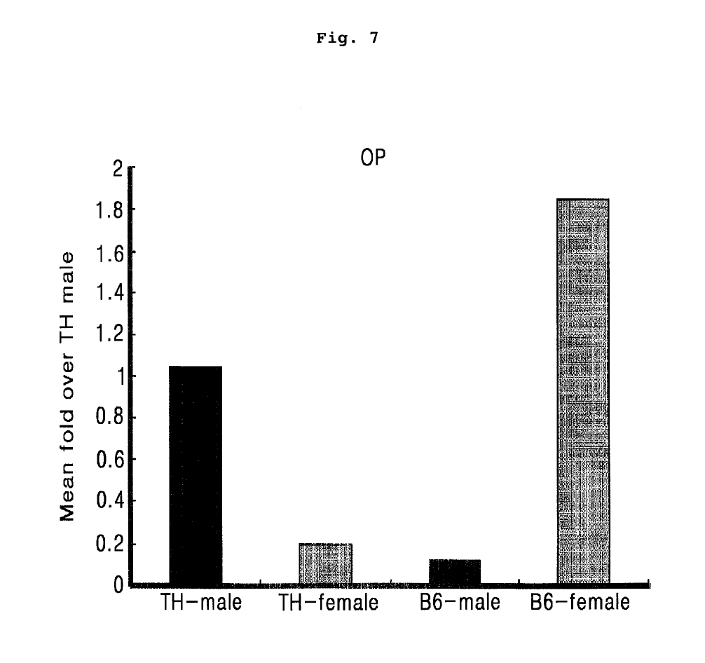

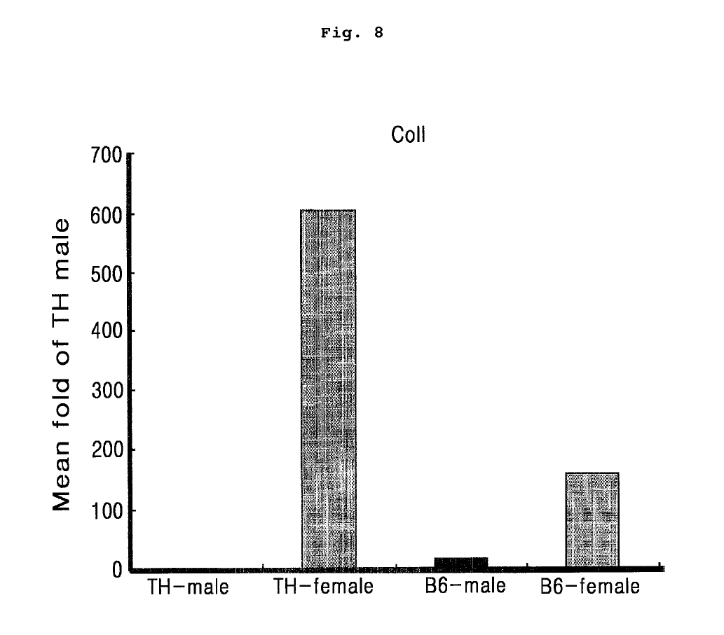

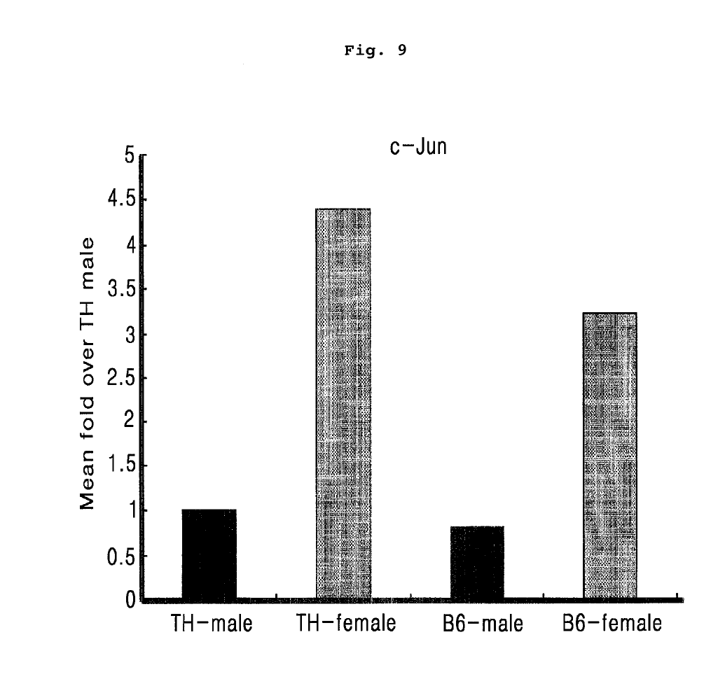

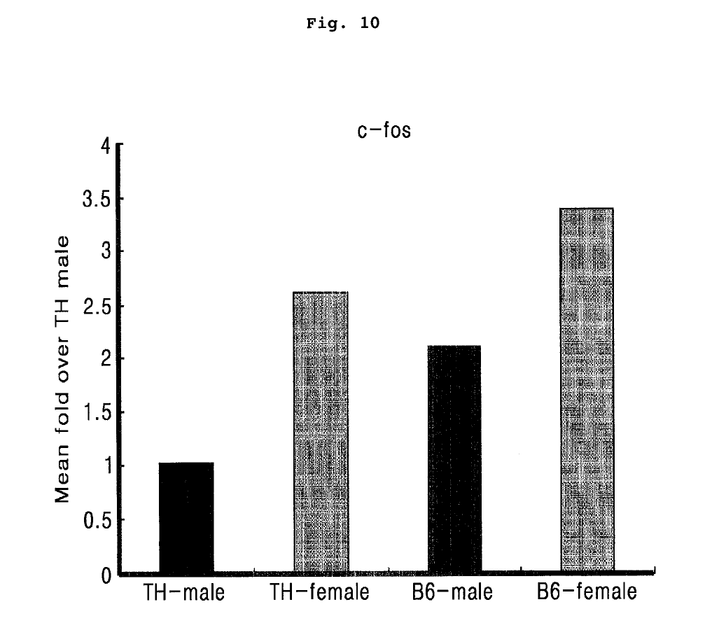

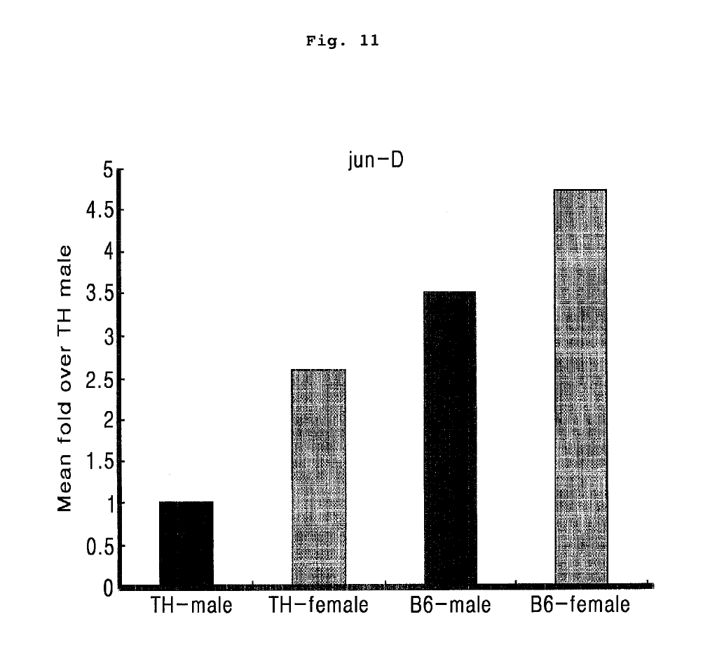

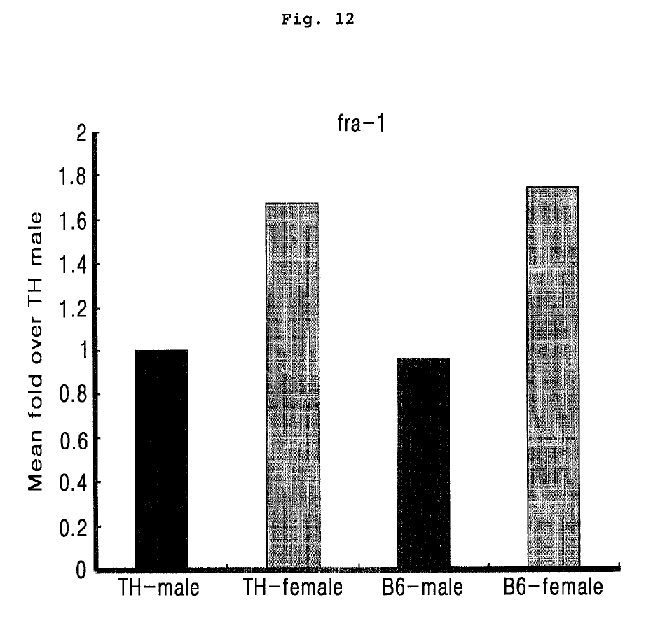

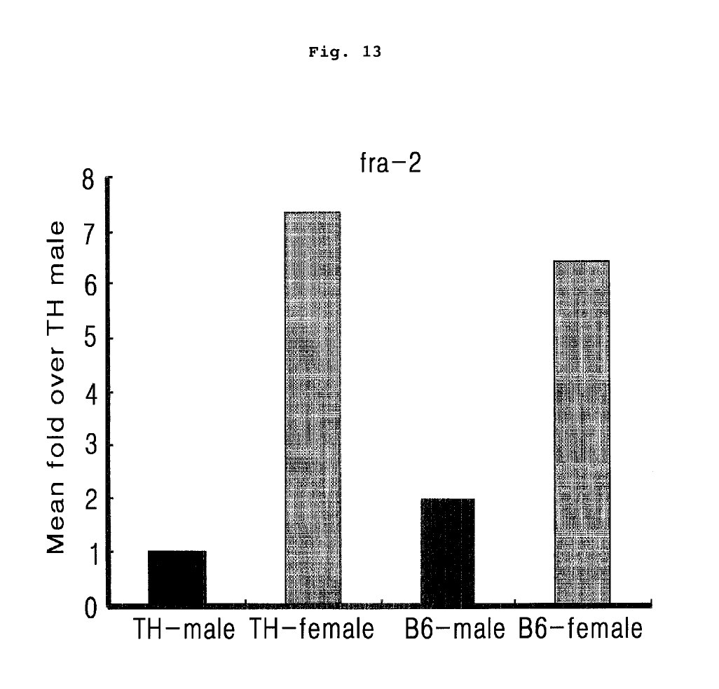

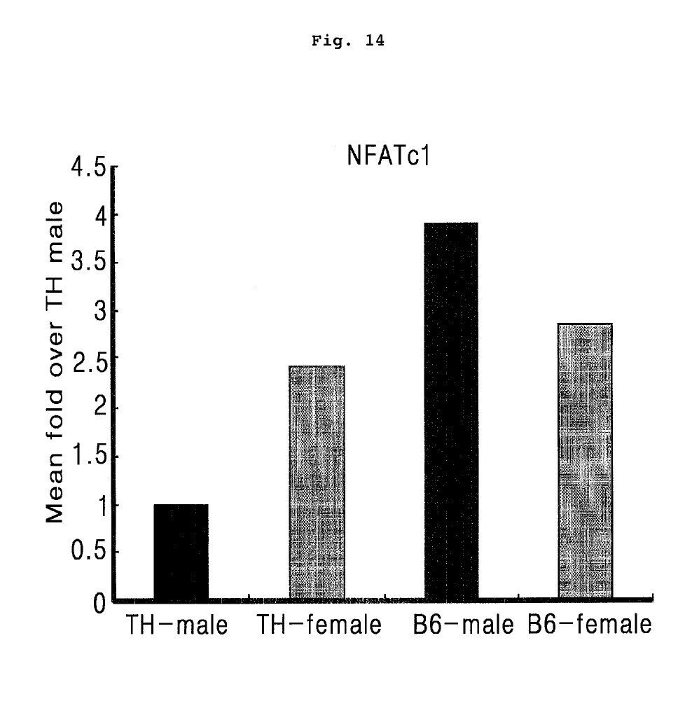

Interestingly, as shown in FIGS. 6 to 14, in the TH mice, the gene expressions of the alkaline phosphatase, which is an initial factor of osteoblast differentiation and Fra2 (FIG. 13), NF-AT1 (FIG. 14), JunD (FIG. 11), and Fos genes (FIG. 10), which are transcription factors for controlling the differentiation of the osteoblast, were significantly decreased. From the result, it can be seen that the bone loss was induced by the complex loss of a variety of genes related to the bone formation.

### Example 7

### In Vivo Effect of Alendronate Using TH Mice

4-week-old male TH mice were orally administered with alendronate (CALBIOCHEM, USA), which is widely used as a osteoporosis medicine, with a concentration of 5 mg/kg, once a day for 4 weeks. Thereafter, the bone mineral density and the bone mineral content of the femur and the thickness and the bone mineral density of the skull were measured as described in Examples 2 and 3.

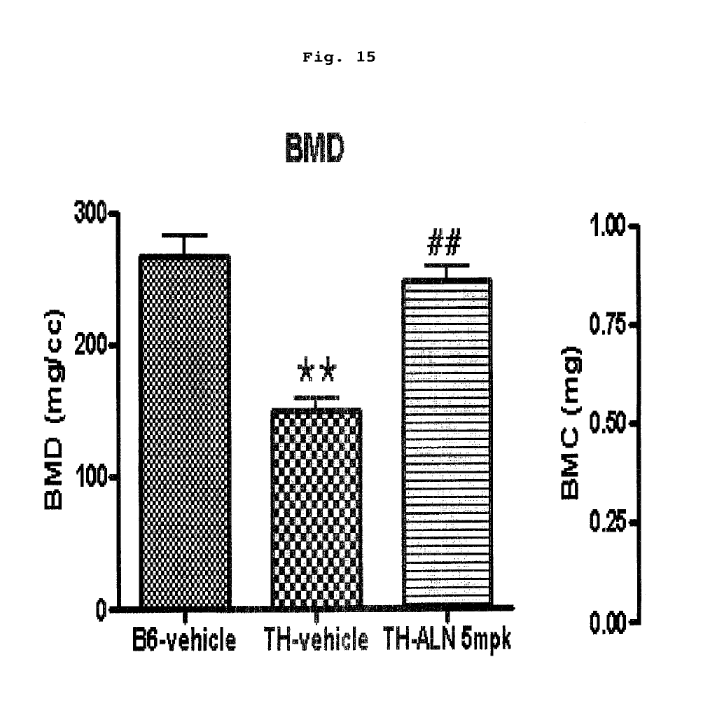

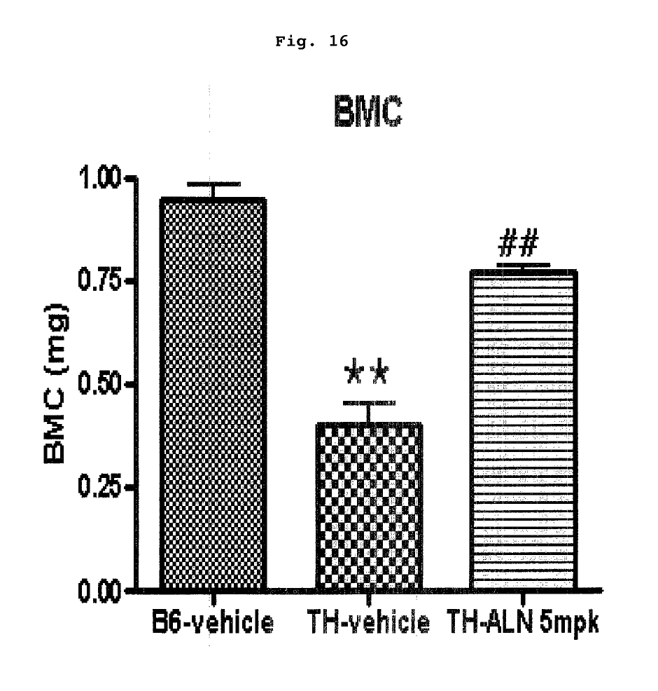

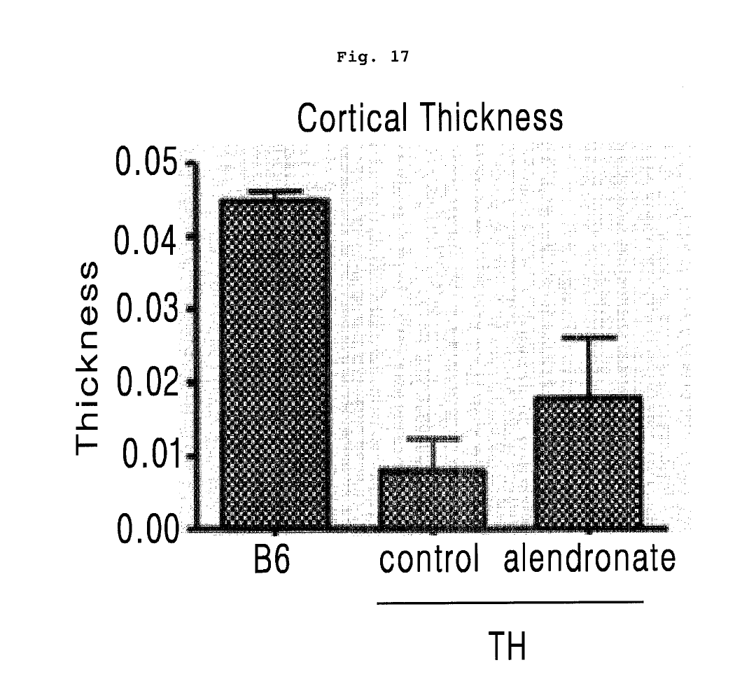

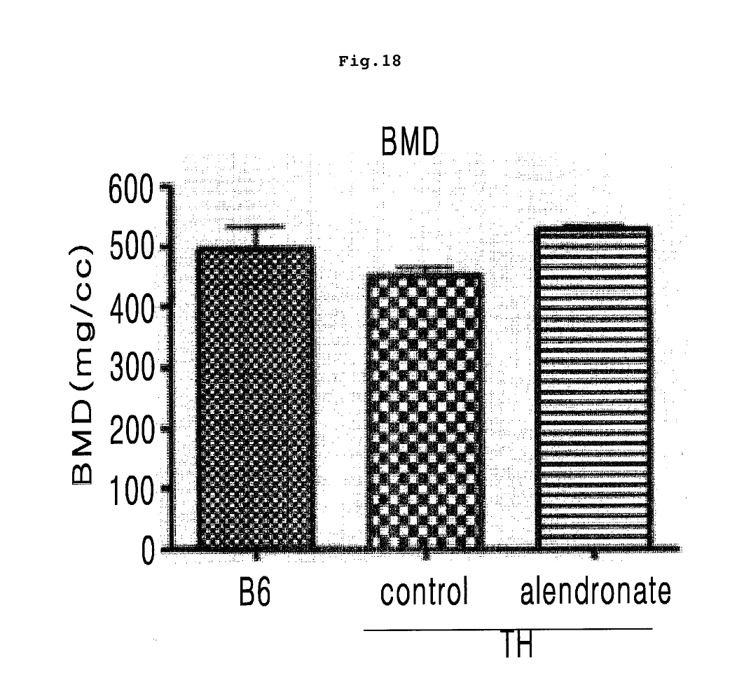

Resultantly, as shown in FIGS. 15 and 16, the TH mice treated with the alendronate significantly recovered their bone mineral densities and bone mineral contents in the femurs in comparison with the 8-week-old TH mice treated with only a vehicle. In addition, the thicknesses and the bone mineral densities of the skulls were significantly recovered in comparison with the TH mice vehicle (see FIGS. 17 and 18).

Meanwhile, an accurate histomorphological scanning was carried out to observe the effect of the alendronate on recovering the bone loss in the femur of the 8-week-old TH mouse. The TH mice and B6 mice were sacrificed with carbon dioxide. The femurs thereof were extracted and scanned 400 times with a thickness of 27 μM using eXplore Locus micro-CT (GE Healthcare, USA) which is devised as a tomographic apparatus for small animals. The scanned images were accurately reformed using Microview (GE Healthcare, USA) to obtain final histomorphological images of the femurs.

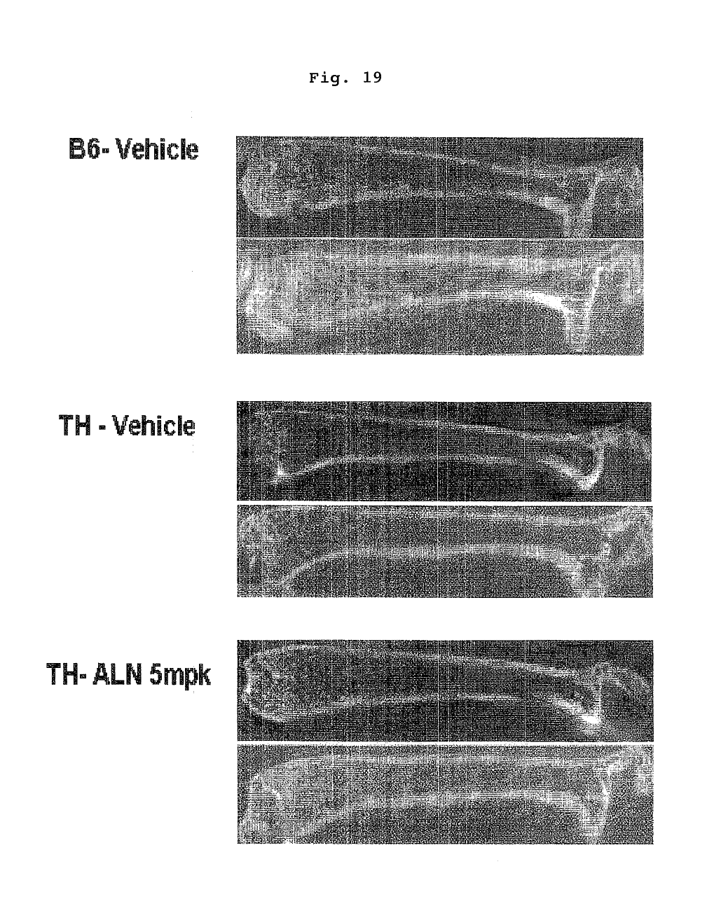

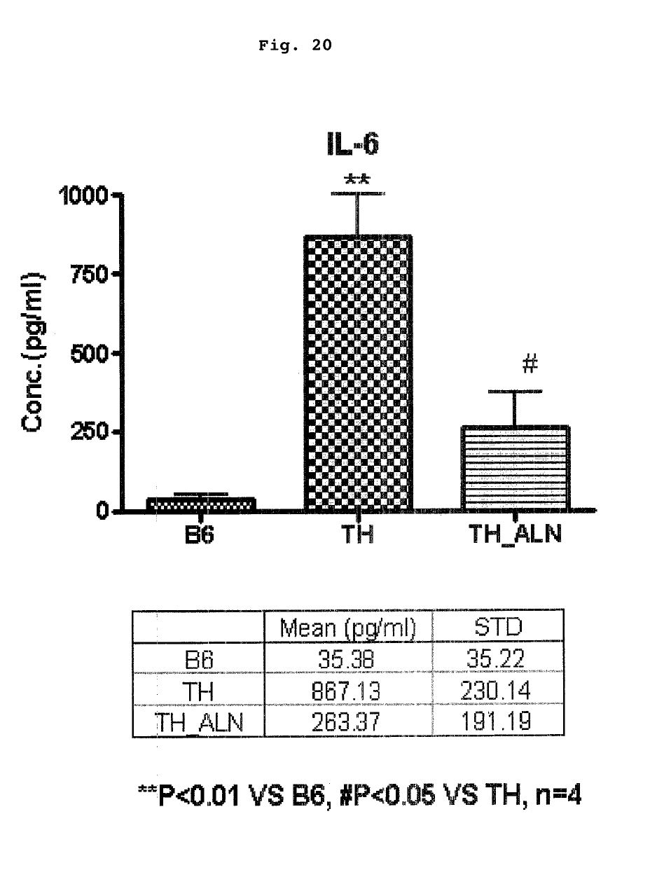

Resultantly, as shown in FIG. 19, the mice group treated with the alendronate was found as being filled with trabeculars.

Meanwhile, after separating the blood serum, IL-6 level was measured using ELISA Kit (ALPCO diagnostics, USA). The measurement showed low IL-6 level in the mice group treated with the alendronate.

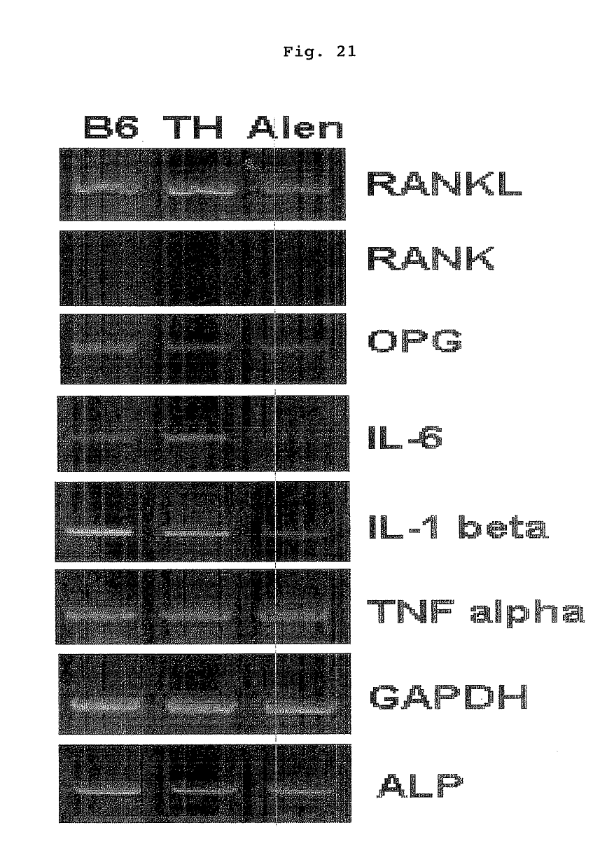

In order to observe the expression of the genes, cells were separated from the bone marrow and cultured for days. Thereafter, the variations of the genes were investigated through RT-PCR as described in Example 5. As shown in FIG. 21, the cells separated from the mice group treated with the alendronate showed an increased OPG and a decreased RANKL in comparison with the vehicle.

From the above mentioned results, it can be seen that the male TH mouse can be used as a new animal model in developing the osteoporosis medicine.

### Example 8

### In Vivo Effect of PTH Using TH Mice

8-week-old male TH mice were orally administered with parathyroid hormone (hPTH(1-34), Sigma, USA), which is started to be used as an osteoporosis medicine increasing the bone mineral content, with a concentration of 50 μg/kg, once a day for 4 weeks. Thereafter, the bone mineral density and the bone mineral content of the femur was measured as described in Example 2.

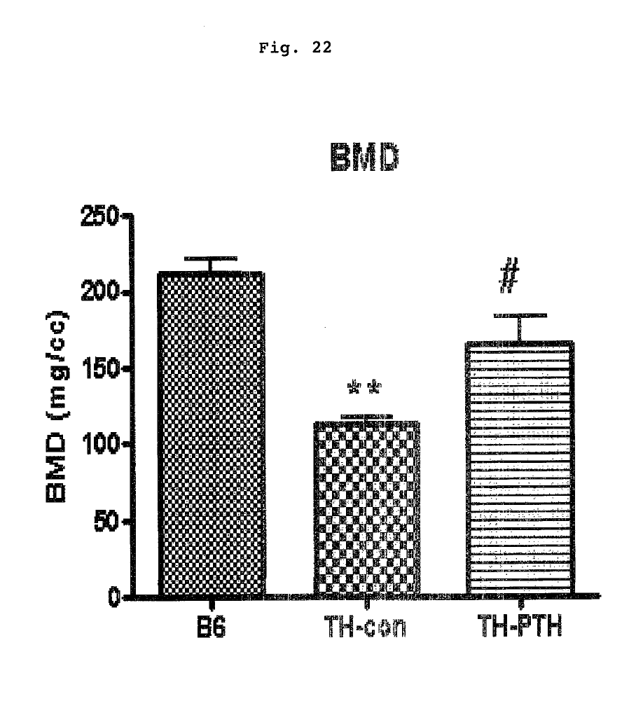

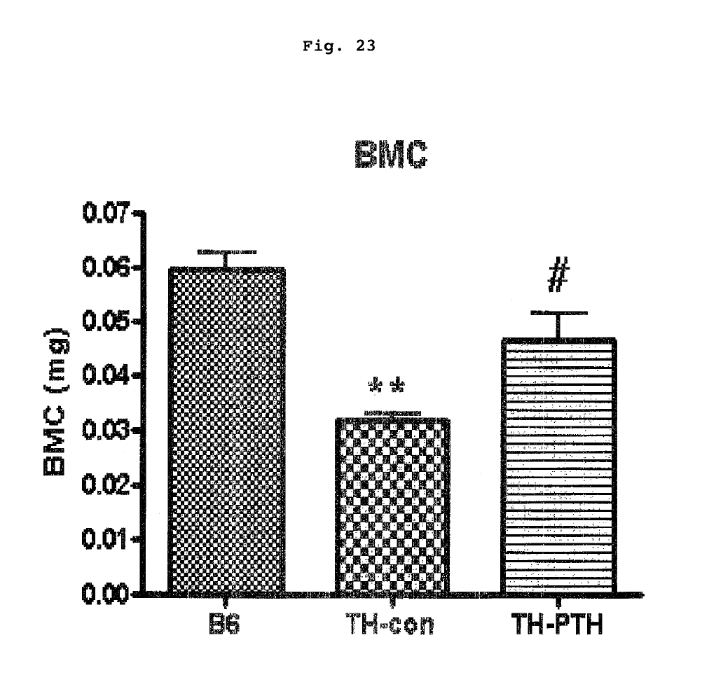

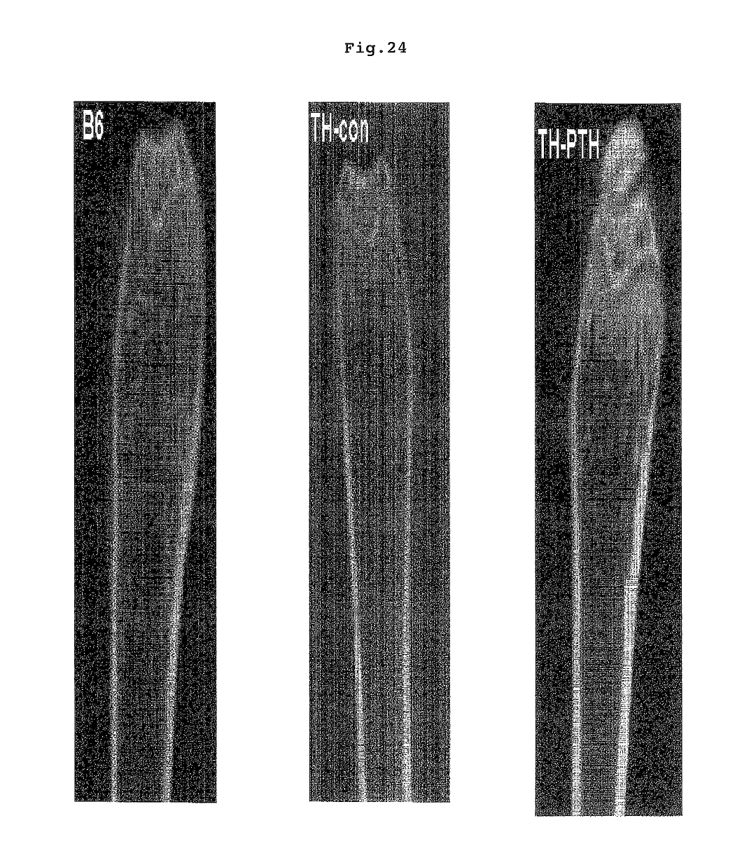

The result showed that, as shown in FIGS. 22 to 24, the TH mice group treated with the PTH significantly recovered the bone mineral densities and the bone mineral contents in comparison with the 12-week-old TH mice group treated with only the vehicle.

From the above mentioned results, it can be seen that the male TH mouse can be used as a new animal model in developing the osteoporosis medicine.

1. A screening method for identifying an agent for treating or preventing osteoporosis and metabolic bone disease, the method comprising:
1) administering a candidate material for the agent for treating or preventing osteoporosis and metabolic bone disease to the male TALLYHO/JngJ (TH) mouse;
2) measuring an index related to osteoporosis and metabolic bone disease from the mouse administered the candidate material of the step 1); and
3) selecting the candidate material that varies the index with significance in the mouse administered the candidate material in comparison with a control that is not administered the candidate material.

2. The screening method as set forth in claim 1, wherein the male TH mouse has an osteoporosis symptom.

3. The screening method as set forth in claim 2, wherein the osteoporosis symptom comprises one or more characteristics selected from a group consisting of decreases of bone mineral density and bone mineral content in a femur, decreases of bone mineral density and bone thickness in a skull, an osteoclast activation rate faster than an osteoblast activation rate, decrease of expression of osteoprotegerin (OPG) gene, increase of expression of receptor activator of NF-κB ligand (RANKL) gene, increase of IL-6, decreases of expressions of Fra2, NF-AT1, JunD and Fos genes, decreases of expressions of alkaline phosphatase and COLL I genes.

4. The screening method as set forth in claim 1, wherein the candidate material of step 1) comprises a peptide, a protein, a nonpeptide compound, a synthetic compound, a fermentation product, a cell extract, a plant extract, an animal texture extract, or a blood plasma.

5. The screening method as set forth in claim 1, wherein the index related to the osteoporosis of step 2) comprises at least one selected from a group consisting of increases of a bone mineral density and a bone mineral content in a femur, increases of a thickness and a bone mineral density in a skull, decrease of IL-6 in a blood serum, increase of expression of an osteoprotegerin (OPG) gene, decrease of expression of a receptor activator of NF-κB ligand (RANKL) gene, increases of expressions of Fra2, NF-AT1, JunD and Fos genes which are transcription factors of an osteoblast, and increases of expressions of alkaline phosphatase (ALP) and COLL I genes which are differentiation factors of the osteoblast.

6. The screening method as set forth in claim 1, wherein the osteoporosis and metabolic bone disease comprises at least one selected from a group consisting of a pre- or post-menopausal osteoporosis of a woman, a senile osteoporosis, an osteoporosis of a man, an osteoporosis after an implantation, an osteoporosis after a surgery, a secondary osteoporosis caused by an osteomalacia and a steroid, and an osteoarthritis.

7. The screening method as set forth in claim 6, wherein the surgery comprises a heart valve surgery or a gastrectomy.
